(19) United States
(12) Patent Application Publication
(10) Pub. No.: US 2008/0260852 A1
(43) Pub. Date: Oct. 23, 2008

(54) SUPERCRITICAL FLUID EXTRACTION PRODUCED BY IN-LINE HOMOGENIZATION

(75) Inventors: Pratibhash Chattopadhyay, North Royalton, OH (US); Boris Y. Shekunov, Aurora, OH (US); K. Adam Gibson, Fairlawn, OH (US)

Correspondence Address: RANKIN, HILL & CLARK LLP, 925 EUCLID AVENUE, SUITE 700, CLEVELAND, OH, 44115-1405, US

(73) Assignee: FERRO PFANSTIEHL LABORATORIES, INC., Waukegan, IL (US)

(21) Appl. No.: 12/017,414

(22) Filed: Jan. 22, 2008

Related U.S. Application Data
(60) Provisional application No. 60/886,165, filed on Jan. 23, 2007.

(51) Int. Cl. A61K 9/14

(52) U.S. Cl. 424/501

(57) ABSTRACT

A method for producing micro and/or nanoparticles by contacting an emulsion with a supercritical fluid. The method can be used to produce an aqueous suspension of particles that include a drug encapsulated in a polymer. The drug and polymer are dissolved in an organic solvent to form an organic solution. The organic solution is pumped into contact with an aqueous solution in an in-line homogenization device to form an emulsion. Immediately after the emulsion in the in-line homogenization device, the emulsion contacts the supercritical fluid. The supercritical fluid extracts the organic solvent from the oil droplets of the emulsion, causing the drug and polymer to precipitate into the aqueous phase of the emulsion and thereby form an aqueous suspension of particles that include a drug encapsulated in a polymer.

### BACKGROUND OF INVENTION

1. Field of Invention

The present invention relates to the production of polymeric micro and nanoparticles via the supercritical fluid extraction of emulsions.

2. Description of Related Art

Chattopadhyay et al., U.S. Pat. Nos. 6,998,051 B2 (hereinafter referred to as “the '051 patent”) and 6,966,990 B2 (hereinafter referred to as “the '990 patent”), both of which are owned by the assignee of the present application, disclose methods for producing aqueous suspensions of micro and/or nanoparticles by extracting organic solvents from emulsions using a supercritical fluid. The processes disclosed in the '051 and '990 patents, both of which are hereby incorporated by reference in their entirety, produce excellent results. However, additional research has resulted in the development of useful modifications, which are disclosed herein.

### BRIEF SUMMARY OF THE INVENTION

In accordance with the present invention, an emulsion is formed between an organic solution that comprises a polymer dissolved in an organic solvent (the organic solution is sometimes referred to herein as the “oil phase”) and an aqueous solution (the aqueous solution is sometimes referred to herein the “water phase”). The aqueous solution forms the continuous phase of the emulsion and the organic solution forms the discontinuous phase of the emulsion (i.e., an oil-in-water emulsion). In the present invention, the emulsion is formed immediately before it contacts a supercritical fluid, which extracts the organic solvent from the organic solution to cause the polymer to supersaturate and precipitate into the aqueous solution in the form of micro or nanoparticles. In accordance with the present invention, the emulsion is formed using an in-line homogenization device that forms the emulsion immediately before it makes contact with the supercritical fluid. Thus, the organic solution and aqueous solution are mixed together, homogenized to form an emulsion, mixed with the supercritical fluid and then injected into an extraction vessel that is maintained at a temperature and pressure above the critical point of the supercritical fluid. The mixture of the emulsion and supercritical fluid is injected into the extraction vessel through a nozzle, which disperses the mixture as a fine spray.

The foregoing and other features of the invention are hereinafter more fully described and particularly pointed out in the claims, the following description setting forth in detail certain illustrative embodiments of the invention, these being indicative, however, of but a few of the various ways in which the principles of the present invention may be employed.

### BRIEF DESCRIPTION OF THE DRAWINGS

FIG. 1 is a schematic process flow diagram of the improved method of the invention.

FIG. 2 is a graph plotting the mean average particle diameter of particles produced in Example 1 as a function of supercritical fluid flow rate.

FIG. 3 is a graph showing the particle size distribution of particles produced in Example 2.

FIG. 4 is a graph showing the particle size distribution of particles produced in Example 3.

FIG. 5 is a scanning electron micrograph of the particles produced in Example 3.

### DETAILED DESCRIPTION OF THE INVENTION

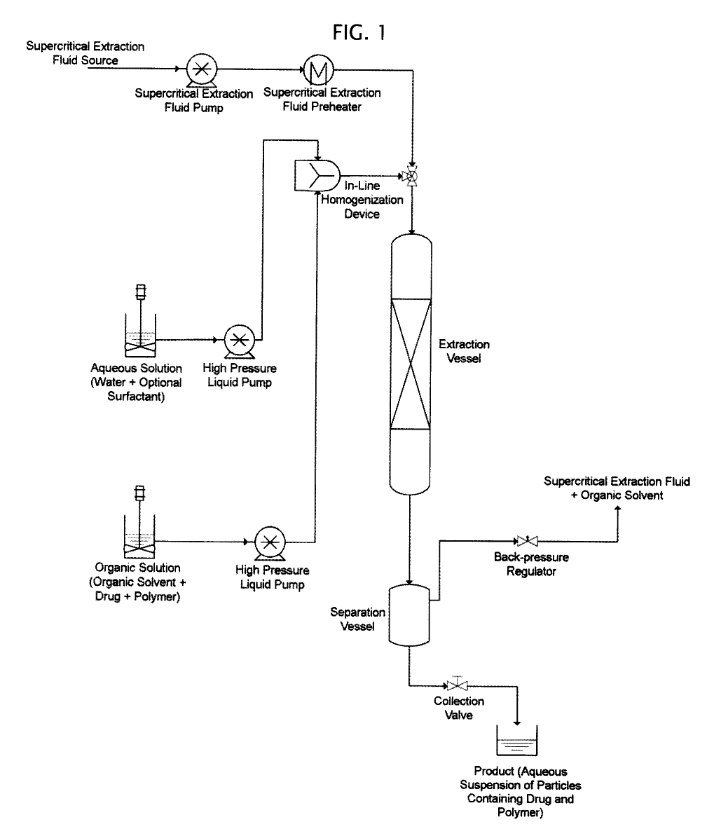

As noted above, the present invention provides certain useful modifications of the processes disclosed in the '051 and '990 patents, both of which have been incorporated by reference in their entirety. Reference is hereby made to FIG. 1, which is a schematic process flow diagram of the improved method of the invention.

In the method of the present invention, the oil phase and the water phase of the emulsion (i.e., the organic solution and the aqueous solution, respectively) are mixed and homogenized using an in-line homogenization device such as an in-line mixer immediately prior to the time the emulsion contacts the supercritical fluid and forms a mixture that is injected into the extraction vessel. It is advantageous for the emulsion to be formed as close in time to the moment when it contacts the supercritical fluid as possible. Thus, the outflow port through which the effluent from the in-line homogenization device exits should be positioned as close to the point where the emulsion contacts the supercritical fluid as possible.

In the present invention, a material that is to be formed into micro or nanoparticles (e.g., a biodegradable polymer, a pharmaceutically active compound, an excipient and/or mixtures of two or more thereof) is dissolved in an organic solvent such as dichloromethane to form an organic solution. The organic solution, when homogenized with an aqueous solution, becomes the internal, discontinuous “oil” phase of the “oil-in-water” emulsion. It will be appreciated that the composition and concentration of the material(s) present in the organic solution, as well as the solubility of the material(s) in the organic solvent, will affect the emulsion's stability and the size of the emulsion droplets.

As in the '051 and '990 patents, a separate aqueous solution is formed that when homogenized with the organic solvent, becomes the external, continuous “water” phase of the “oil-in-water” emulsion. The aqueous solution can consist of water only, but preferably further comprises one or more stabilizers, surfactants, and/or excipients, which are dissolved or suspended in the water. The concentration of stabilizer(s), surfactant(s) and/or excipients can be adjusted to yield the desired emulsion droplet size, which directly correlates to the particle size of the final product (i.e., the precipitated particles suspended in the aqueous solution).

In the method of the present invention, the organic solution and the aqueous solution that are mixed together to form the emulsion are co-introduced into an in-line homogenization device by means of high-pressure liquid pumps immediately prior to contacting the supercritical fluid. The in-line homogenization device can be a static mixing element or multiple static mixing elements in series. Alternatively, the homogenization device could comprise a tank with a high shear mixer, or other high-pressure homogenization apparatus, provided that the formation of the emulsion occurs very close in time (e.g., less than 5 seconds, and more preferably less than 2 seconds) prior to the moment when the emulsion contacts the supercritical fluid. The relative rate at which the organic solution and the aqueous solution are co-introduced into the homogenization device influences the characteristics of the final emulsion.

As in the methods described in the '051 and '990 patents, an extracting fluid is supplied in a supercritical state by means of a specialized high-pressure pump and a heat exchanger, which control the temperature and pressure on the extracting fluid, respectively. In the preferred embodiment of the invention, the extracting fluid is supercritical carbon dioxide (“SC CO₂”).

The effluent from the outlet side of the in-line homogenization element contacts the supercritical extracting fluid immediately past the in-line homogenization device. The emulsion and supercritical extracting fluid are mixed together and immediately introduced into the extraction column through a nozzle. The nozzle, together with the high pressure extracting fluid, serves to disperse the homogenized emulsion in the supercritical extracting fluid to facilitate efficient extraction. The extraction column is pressure and temperature controlled such that the supercritical extracting fluid is maintained in its supercritical state.

In the extraction column, the organic solvent present in the internal phase of the emulsion is extracted into the supercritical extracting fluid, causing the material(s) dissolved in the organic solvent to supersaturate and thus precipitate in the form of micro or nano-sized particles into the aqueous solution (external phase). The precipitated particles tend to be spherical in shape. The external phase of the emulsion is not extracted by the SC CO₂. Therefore, the particles become suspended in the external phase, forming an aqueous suspension of particles of the material.

The particulate suspension and the organic solvent-laden SC CO₂ exit the extraction column and flow into a separation vessel. The organic solvent-laden SC CO₂ exits through the top of the separation vessel. The aqueous suspension of particles settles to the bottom via the force of gravity, where it is removed through a collection valve. The collection valve serves to regulate the flow of product suspension, thereby maintaining the desired level of liquid in the extraction column, preventing system depressurization.

The effluent from the extraction column enters the separation vessel through a tube that protrudes into the vessel (e.g., a dip tube). This minimizes loss of product suspension through the SC CO₂ outlet by providing physical separation between the inflow of the SC CO₂/emulsion mixture and outflow of organic solvent laden SC CO₂. System pressure is controlled by means of a back-pressure regulator on the organic solvent laden SC CO₂ vent line.

The method of the present invention is particularly suitable for producing micro and/or nanoparticles of pharmaceutical compositions that comprise one or more drugs encapsulated in a biodegradable polymer. In such applications, a polymer such as poly(lactic-co-glycolic) acid (“PLGA”) is preferable dissolved in a suitable organic solvent such as dichloromethane to form an organic solution. The concentration of the polymer in the organic solvent can be adjusted throughout a broad range, but a concentration of about 10% by weight is preferred. The drug to be encapsulated in the polymer is also dissolved in the organic solvent. The concentration of the drug in the organic solvent will be dictated by the amount to be delivered and other considerations. In many applications, a concentration of the drug of about 1% by weight is suitable.

As noted, PLGA is a preferred polymer for drug delivery applications, but other organic solvent soluble polymers can be used. Suitable polymers include various molecular weights and G/L ratios for PLGA, PLGA-polyethylene glycol (“PEG”) composites, Eudragit polymers, ethyl cellulose polymers, and other polymers that are substantially insoluble in water, and soluble in an appropriate organic solvent.

A preferred organic solvent is dichloromethane. However, other preferred organic solvents include chloroform and ethyl acetate. Virtually any organic solvent that is capable of dissolving the drug and the polymer to a sufficient degree, and which is soluble in the supercritical fluid, and which is not significantly miscible with water can be used.

The concentration of the polymer in the organic solvent is preferably within the range of between about 5% to about 20% by weight of the organic solution, but could be >0 to 50% by weight of the organic solution, if desired. The drug concentration is preferably within the range 1-10% by weight of the organic solution, but could be >0 to 30% by weight organic solution, if desired. Drug loading is determined by the relative amounts of polymer and drug, and could range from about 1% to about 90% by weight of the polymer (in the final particles). Preferred drug loading is from about 5% to about 20% by weight of the polymer.

The aqueous solution can comprise only water, but for drug applications, the aqueous solution preferably further comprises a suitable surfactant and/or stabilizer such as polyvinyl alcohol (“PVA”). When PVA is used, a concentration of about 3% by weight of the aqueous solution is preferred. It will be appreciated that the concentration of the surfactant and/or stabilizer is not critical, but the least amount necessary to form the emulsion should be used.

The surfactants and/or stabilizers ensure that the emulsion is sufficiently stable during the extraction step to ensure that particles with the desired characteristics (e.g., size) are formed during the extraction step. As noted, PVA is the preferred stabilizer for drug applications, but other surfactants/stabilizers such as polysorbate, sorbitan monooleate, sodium dodecyl sulfate, Vitamin E (d-alpha-tocopheryl), polyethylene glycol 1000 succinate, Tyloxapol, and other surface active compounds can be used. The concentration of surfactants and/or stabilizers can range from 0% to the solubility limit of the compound(s) in water.

The extraction vessel is preferably pressurized with supercritical fluid such as SC CO₂ before the emulsion in contact with the supercritical fluid is pumped into the extraction vessel. The SC CO₂ can be pumped into the extraction vessel through a low dead-volume mixing element, which can comprise a simple intersection or “tee.” It could also be a more complex static mixing element of any design. As noted, the preferred extraction fluid is SC CO₂, but other supercritical fluids such as supercritical ethane, supercritical ethylene, supercritical propane, supercritical propylene and supercritical pentane, for example, could be used if desired.

The pressure and temperature of the extraction vessel must be maintained such that the supercritical fluid is maintained above its critical point during the extraction step. For SC CO₂, the extraction vessel temperature is preferably about 35° C., which is slightly above the critical temperature of SC CO₂. The suitable temperature range for an extraction vessel charged with SC CO₂ is about 32° C. to about 95° C.

The pressure of the extraction vessel must also be maintained to keep the supercritical fluid in a supercritical state. For SC CO₂, the preferred pressure is about 80 bar (slightly above the critical pressure for SC CO₂), which can be maintained through the use of a back pressure regulator downstream of the collection vessel as shown in FIG. 1. The permissible pressure range for SC CO₂ is about 80 to about 600 bar.

The organic solution and the aqueous solution are co-introduced into the in-line homogenization device using suitable pumps. The flow rates of the pumps should correspond to the desired proportions of the internal and external phases of the emulsion. For example, a 20% internal phase emulsion can be formed by simultaneously flowing the organic solution at a flow rate of 2 ml/min and the aqueous solution at a flow rate of 8 ml/min into the homogenization device. The amount of the internal (oil) phase comprising the emulsion is preferably about 5% to about 30% by weight of the emulsion, but could range from about 1% to about 50% by weight of the emulsion.

The combined flow rate of the emulsion (i.e., the flow rate of the organic solution plus the flow rate of the aqueous solution) is preferably less than the flow rate of the SC CO₂ (e.g., 10%, which equates to a flow rate ratio of 1:9). The preferred flow rate ratio of emulsion to SC CO₂ is preferably from about 1:5 to about 1:30, but could be as high as 1:100.

Once the extraction vessel has been pressurized with the supercritical fluid, the emulsion and supercritical fluid are pumped into contact with each other immediately after the emulsion exits from the in-line homogenization device and are thereby mixed and then injected into the extraction vessel. After several minutes, an aqueous suspension of particles will begin to form. If a polymer and a drug are dissolved in the organic solution, the particles will include the drug dispersed or encapsulated in the polymer. The aqueous suspension of particles can be collected using a port and valve at the bottom of the collection vessel, as shown in FIG. 1. Adjustments can be made so that the collection flow rate is approximately equal the rate of product suspension generation (i.e., equal to the inflow rate of emulsion/supercritical fluid). For small scale processing apparatus, it is sometimes necessary to limit the collection flow rate in order to prevent the collection line from freezing. The rate of product collection can be manually or computer controlled.

It will be appreciated that the method of the present invention allows for the production of micro and/or nanoparticles comprising a drug encapsulated in a polymer in a continuous process as opposed to a batch process. The process is highly scalable, such that nearly any rate of product generation can be achieved simply by changing the apparatus slightly.

In many cases, the use of an in-line homogenization device surprisingly results in the production of particles that have different characteristics than particles produced using an emulsion that is prepared in advance of the extraction step. For example, particles produced from emulsions that are prepared more than a few moments prior to contact with the supercritical extraction fluid can undergo premature hardening. In addition, there is a greater risk of a loss of particle size control and a loss of material to be encapsulated when the emulsion is prepared using a method other than an in-line homogenization device. Pre-formation and homogenization of emulsions prior to processing also requires significant formulation work in order to develop an emulsion that does not phase separate before or during extraction. In the method of the present invention, the components of the emulsion are mixed together, homogenized, and co-injected into the extraction vessel with the supercritical extraction fluid within a few seconds, which renders emulsion stability issues less consequential. As such, the useful range for parameters such as drug concentration and amount of internal phase is expanded, thereby allowing higher drug loading, greater process yield, and better encapsulation efficiency.

In addition, because the emulsion is formed immediately prior to contact with the supercritical extraction fluid, the particle size can be easily controlled simply by adjusting the flow rate of the supercritical extraction fluid relative to the flow rate of emulsion. The supercritical fluid, at higher flow rates, provides a dispersion force that can further break the emulsion into substantially uniform droplets, which yields smaller particle sizes.

The following examples are intended to illustrate the invention without limiting it in any way.

### EXAMPLE 1

Using an apparatus configured as shown in FIG. 1, an aqueous solution consisting of 3% w/w PVA in water was pumped to a static mixer and therein homogenized with an organic solution consisting of 10% w/w PLGA in dichloromethane (“DCM”) to form an emulsion. The ratio of the flow rate of the organic solution into the static mixer to the flow rate of the aqueous solution into the static mixer was maintained at about 1:4.

Immediately upon exiting the static mixer, the emulsion was mixed with a flow of SC CO₂ and injected into an extraction column configured as shown in FIG. 1. The ratio of the flow rate of the emulsion into the extraction column to the flow rate of the SC CO₂ into the extraction column was maintained at 1:10. The temperature in the extraction column was maintained at about 35-40° C., and the pressure in the extraction column was maintained at about 80 bar.

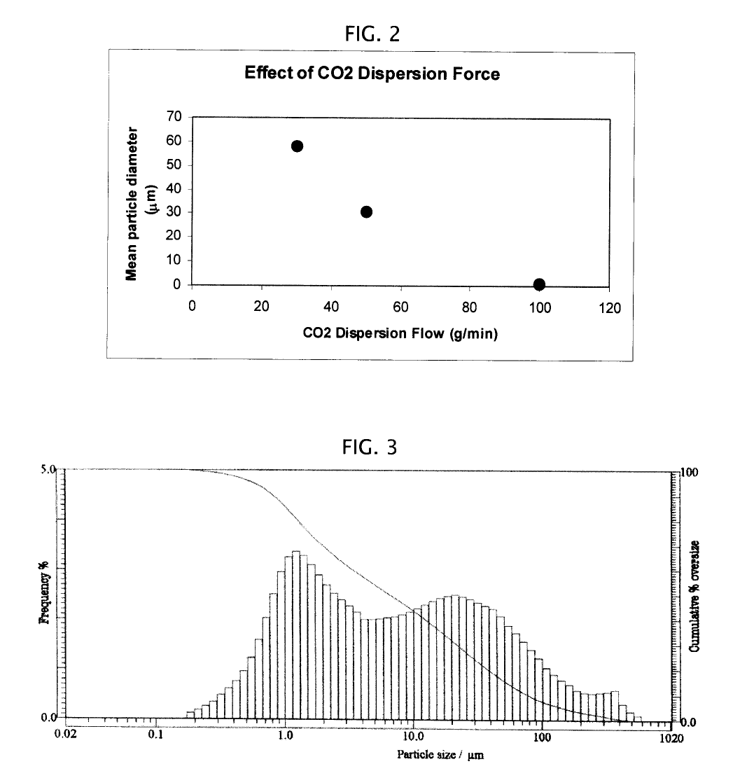

In the extraction column, the SC CO₂ extracted the DCM from the discontinuous oil droplets in the emulsion thereby causing the PLGA to supersaturate and precipitate into the continuous aqueous phase and thereby form an aqueous suspension of precipitated particles. FIG. 2 is a graph that shows the mean average particle diameter of the particles as a function of the SC CO₂ flow rate.

### EXAMPLE 2

Using an apparatus configured as shown in FIG. 1, an aqueous solution consisting of 3% w/w PVA in water was pumped to a static mixer and therein homogenized with an organic solution consisting of 10% w/w PLGA in DCM to form an emulsion. The flow rate of the organic solution into the static mixer was maintained at about 1.0 ml/min. The flow rate of the aqueous solution into the static mixer was maintained at 4.0 ml/min. Thus, the flow ratio was about 1:4.

Immediately upon exiting the static mixer, the emulsion was mixed with SC CO₂, which was flowing at a rate of 50 g/min, and injected into an extraction column configured as shown in FIG. 1. Thus, the flow ratio of emulsion to supercritical fluid was about 1:10. The temperature in the extraction column was maintained at about 37° C., and the pressure in the extraction column was maintained at about 80 bar.

In the extraction column, the SC CO₂ extracted the DCM from the discontinuous oil droplets of the emulsion thereby causing the PLGA to supersaturate and precipitate into the continuous aqueous solution phase of the emulsion thereby forming an aqueous suspension of precipitated particles. FIG. 3 is a graph showing the particle size distribution of particles.

### EXAMPLE 3

Using an apparatus configured as shown in FIG. 1, an aqueous solution consisting of 3% w/w PVA in water was pumped to a static mixer and therein homogenized with an organic solution consisting of 10% w/w PLGA and 1% w/w/ budesonide in DCM to form an emulsion. The flow rate of the organic solution into the static mixer was maintained at about 0.6 ml/min. The flow rate of the aqueous solution into the static mixer was maintained at 2.4 ml/min. Thus, the flow ratio of organic solution to aqueous solution was 1:4.

Immediately upon exiting the static mixer, the emulsion was mixed with SC CO₂, which was flowing at a rate of 50 g/min, and injected into an extraction column configured as shown in FIG. 1. Thus, the flow ratio of the emulsion to the supercritical fluid was about 1:16.67. The temperature in the extraction column was maintained at about 37° C., and the pressure in the extraction column was maintained at about 80 bar.

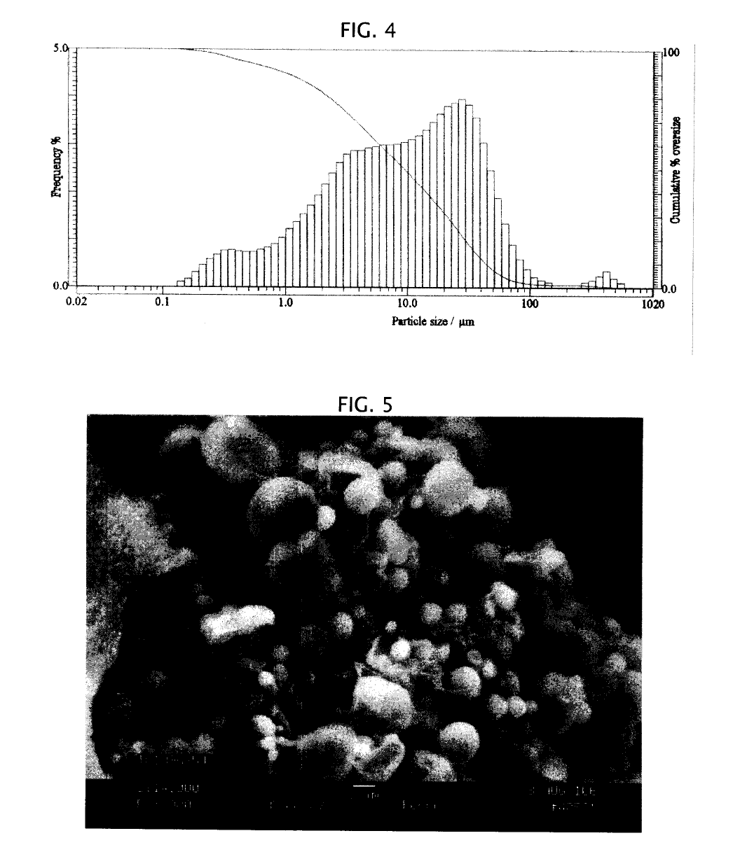

In the extraction column, the SC CO₂ extracted the DCM from the discontinuous oil droplets of the emulsion thereby causing the PLGA to supersaturate and thus precipitate into the continuous aqueous phase in the form of small particles, thereby forming an aqueous suspension of particles. FIG. 4 is a graph showing the particle size distribution of particles. FIG. 5 is a scanning electron micrograph of the particles.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. A method for producing an aqueous suspension of particles comprising:
preparing an aqueous solution;
preparing an organic solution comprising a polymer and a drug dissolved in an organic solvent that is not miscible in the aqueous solution;
simultaneously flowing the organic solution and the aqueous solution into an in-line homogenization device to form an emulsion having a continuous phase comprising the aqueous solution and a discontinuous phase comprising the organic solution, wherein the emulsion exits from the in-line homogenization device as an emulsion effluent stream;
contacting the emulsion effluent stream exiting from the in-line homogenization device with a stream of a supercritical extraction fluid to form a mixture;
injecting the mixture into an extraction vessel maintained at a temperature and a pressure sufficient to maintain the supercritical fluid in a supercritical state whereby the supercritical extraction fluid extracts the organic solvent from the discontinuous phase of the emulsion and into the supercritical extraction fluid and thereby precipitates particles comprising the drug and the polymer into the continuous aqueous phase to form the aqueous suspension of particles; and
separating the aqueous suspension of particles and the supercritical extraction fluid containing the extracted organic solvent from each other.

2. The method according to claim 1 wherein the aqueous solution comprises water and one or more surfactants and/or stabilizers.

3. The method according to claim 1 wherein the in-line homogenization device is a static mixer.

4. The method according to claim 1 wherein the in-line homogenization device comprises multiple static mixing elements in series.

5. The method according to claim 1 wherein the in-line homogenization device comprises a tank with a high shear mixer.

6. The method according to claim 1 wherein the in-line homogenization device comprises a high-pressure homogenizer.

7. The method according to claim 1 wherein the emulsion effluent stream from the in-line homogenization device contacts the supercritical extraction fluid within five seconds of the emulsion effluent stream exiting the in-line homogenization device.

8. The method according to claim 1 wherein the emulsion effluent stream from the in-line homogenization device contacts the supercritical extraction fluid within two seconds of the emulsion effluent stream exiting the in-line homogenization device.

9. The method according to claim 1 wherein the particles in the aqueous suspension of particles comprise the drug encapsulated in the polymer.

10. The method according to claim 1 wherein the particles in the aqueous suspension of particles are substantially spherical in shape.

11. The method according to claim 1 wherein the particles in the aqueous suspension of particles comprise from about 1% to about 90% of the drug by weight of the polymer.

12. The method according to claim 1 wherein the particles in the aqueous suspension of particles comprise from about 5% to about 20% of the drug by weight of the polymer.

13. The method according to claim 1 wherein a mean particle size of the particles in the aqueous suspension is selected by adjusting a flow rate of the supercritical extraction fluid relative to a flow rate of the emulsion into the extraction vessel.
